(19) United States
(12) Patent Application Publication
(10) Pub. No.: US 2008/0154343 A1
(43) Pub. Date: Jun. 26, 2008

(54) EUSTACHIAN TUBE TREATMENT SYSTEMS

(76) Inventors: Kasey K. LI, Palo Alto, CA (US); George Y. CHOI, Redwood City, CA (US)

Correspondence Address: LEVINE BAGADE HAN LLP, 2483 EAST BAYSHORE ROAD, SUITE 100, PALO ALTO, CA, 94303, US

(21) Appl. No.: 11/864,741

(22) Filed: Sep. 28, 2007

Related U.S. Application Data
(60) Provisional application No. 60/871,214, filed on Dec. 21, 2006.

(51) Int. Cl. A61N 5/06; A61B 1/00; A61B 17/32; A61B 18/18

(52) U.S. Cl. 607/89; 600/101; 606/15; 606/167

(57) ABSTRACT

Eustachian tube treatment systems are described herein. One method for reducing or removing any obstructions within the Eustachian tube is to apply laser energy to the underlying tissue. One instrument may be used to deliver laser energy and to optionally provide an infusion or injection of a fluid directly into the tissue as well as optionally provide for ultrasound energy application as well. One or more optical fibers which may extend through needles inserted into the tissue may be utilized to deliver the laser energy.

### CROSS-REFERENCE TO RELATED APPLICATIONS

This application claims the benefit of priority to U.S. Prov. Pat. App. 60/871,214 filed Dec. 21, 2006, which is incorporated herein by reference in its entirety.

### FIELD OF THE INVENTION

The present invention relates to devices and methods for treating tissue regions in or around the Eustachian tube of a patient for clearing or reducing tissue obstructions. More particularly, the present invention relates to devices and methods for clearing obstructed tissue regions by reducing sub-mucosal tissue in or around the Eustachian tube.

### BACKGROUND OF THE INVENTION

The Eustachian tube, typically called the pharyngotympanic tube, is a tube that links the pharynx to the middle ear and is typically about 35 mm in length in adults. Normally, the Eustachian tube is closed but it can open to let a small amount of air through to equalize the pressure between the middle ear and the atmosphere. This pressure equalization typically results in a small pop detected by the person and frequently occurs when undergoing changes in air pressure, such as traveling in an air-plane or driving through mountainous regions. The tube may be opened by contracting muscles in the neck, e.g., either by yawning, swallowing, or simply contracting these muscles voluntarily. Equalizing the pressure through the Eustachian tube helps to prevent damage to the middle ear as well as relieving discomfort to the person.

Aside from equalizing pressure, the Eustachian tube also drains mucus from the middle ear. Upper airway infections or allergies can cause the Eustachian tube to become swollen, trapping bacteria and causing ear infections. This swelling can be reduced through the use of drugs, such as pseudoephedrine, or via endonasal therapy to initiate the draining and cleansing in the Eustachian tubes.

However, the Eustachian tube may become obstructed often resulting in a condition known as Eustachian Tube Dysfunction (ETD). In ETD the tube may be swollen shut or collapsed, the ear drum may retract, and fluid may build up in the middle ear adversely affecting hearing as well as becoming infected. ETD often starts after a head cold or sinus problem or may have no obvious cause at all, but may persist for months or even longer. A physician may treat ETD by surgically placing a drainage tube, such as a myringotomy tube or Eustachian tube catheter, to alleviate the condition by allowing air pressure to equalize across the eardrum and also allowing fluid in the middle ear to drain.

However, such procedures require the implantation of the drainage tube through the tympanic membrane as well as drainage of any fluids through the tube. Not only does the procedure lead to discomfort but may also lead to risks in adversely affecting the hearing of the patient.

Accordingly, there exists a need for devices and methods which are efficacious and safe in clearing the obstructed Eustachian tube passageways at least for an extended period of time.

### SUMMARY OF THE INVENTION

By reducing the size of tissue, particularly sub-mucosal tissue, in and/or around the Eustachian tube, obstruction of the Eustachian tube lumen can be reduced to improve the passage of air and fluid therethrough. One method for reducing the tissue involves the application of energy to the tissue regions beneath the surface of the tissue, e.g., ultrasound, laser energy, etc.

One variation of a treatment instrument which may be used to deliver energy, such as ultrasound energy, to the underlying tissue may also be configured to provide an infusion or injection of a fluid directly into the tissue being treated by the ultrasound energy. The fluid injected into the tissue may be used to bulk up the physical size of the tissue by injecting the fluid to present a larger surface area to the ultrasound transducers positioned along the instrument. The enlarged surface area may help to ensure that the ultrasound energy is properly delivered directly into the intended tissue rather than surrounding tissues.

The injected fluid may also be used for drug delivery directly into the treated tissue. For instance, anesthetic fluids or other fluids infused with analgesics may be injected into the tissue to provide for pain management during and after the application of the ultrasound energy. Additionally, other drugs for injection may include any number of medications, such as non-steroidal drugs, anti-inflammatory drugs, anti-bacterial drugs, etc. which may be injected to control excessive post-operative swelling as well as infection. Additionally, the one or more injection needles may be utilized as a positioning tool for ensuring that the ultrasound energy, which is directional, is delivered into the intended tissue. For example, the injection needle(s) may be intially positioned directly within the tissue prior to application of the ultrasound energy since the ultrasound transducer(s) along the probe may be aligned with the injection needle(s). Accordingly, if the needle(s) is positioned directly within the tissue to be treated, the operator may be assured that the ultrasound energy will be directionally aligned with the appropriate tissue region.

The ultrasound and infusion probe may have an elongate shaft which is sufficient to allow for insertion and advancement into the Eustachian tube and against the appropriate tissue surface. The distal end portion may be angled relative to the elongate shaft or it may be straight depending upon the desired configuration. The distal end portion may have an end effector assembly which has one or more hollow infusion/injection needles which are retractably disposed within the distal end portion. During advancement into the Eustachian tube and positioning against the tissue, the infusion/injection needles may be positioned within the distal end portion so as to present a smooth atraumatic surface to the tissue. When a fluid is to be injected into the tissue after the probe has been desirably positioned against the tissue surface, a control or advancement mechanism on handle, which is connected to a proximal end of the shaft, may be actuated to advance the needles at least partially out of the distal end portion. Between or adjacent to the needles are one or more ultrasound transducers along the body of the distal end portion.

An electronic/fluid cable is electrically and fluidly connected to the handle and is further connected to a power/infusion assembly, which may hold a fluid reservoir and a pump electrically coupled to a controller or central processor. Any of the above-mentioned fluids, e.g., analgesics anesthetics, anti-inflammatory drugs, water, saline, etc., may be filled within the reservoir for delivery through the cable and through the one or more infusion/injection needles for delivery into the tissue.

In use, the elongate shaft and distal end portion may be advanced through the patient's ear canal, nostril, or mouth to gain access to the Eustachian tube. The distal end portion of the elongate shaft may be positioned anywhere in or around the Eustachian tube and the infusion/injection needles may be deployed from the distal end portion and pierced into the tissue, where the fluid may be injected and/or infused from the needles into the tissue. As the fluid is injected into the tissue, the infused tissue may begin to expand in size thereby pressing against the distal end portion. The fluid may be stopped and the focused ultrasound energy may then be transmitted from the transducers into the underlying expanded tissue.

Once the injection and ultrasound treatment has been concluded, the damaged underlying tissue may scar and eventually reduce a size of the tissue, thereby resulting in an unobstructed Eustachian tube lumen. The treatments may be performed periodically between extended time periods while the tissue regenerates or on an as-needed basis.

In alternative configurations, the distal end electors may include a mechanism for securely pressing the surface of the elongate shaft against the tissue surface to be treated to ensure piercing of the needles into the tissue as well as sufficient contact for the ultrasound transmission. For instance, expandable balloons and wires or ribbon members which may be reconfigured from a low-profile configuration against the elongate shaft to an expanded shape may be utilized.

Moreover, the ultrasound and infusion probe may optionally include an additional radio-frequency energy generator to deliver RF energy to one or more needles to ablate the pierced tissue. The ultrasound and infusion probe may also optionally include a cooling unit fluidly connected via a fluid line to the power/infusion assembly. Cooled fluid may be fluidly connected through the elongate shaft to a cooling fluid port positioned along the distal end portion.

Additionally, aside from the use of ultrasound transducers for delivering energy to the tissue, laser energy may alternatively be used to facilitate tissue reduction while achieving hemostasis and minimizing tissue injury to surrounding tissue regions.

### BRIEF DESCRIPTION OF THE DRAWINGS

FIG. 1 shows an illustrative view of a Eustachian tube in relation to the middle ear cavity and outer ear of a patient.

FIG. 2 shows a variation of a treatment instrument which may be used to deliver ultrasound energy as well as for providing an infusion or injection of a fluid directly into tissue being treated.

FIGS. 3A and 3B illustrate partial cross-sectional detail views of a distal end portion of the elongate shaft showing the infusion/injection needles positioned within and projected out from the elongate shaft, respectively.

FIG. 3C illustrates a side view of another variation in which the infusion/injection needle may project distally from a distal end of the elongate shaft.

FIG. 4 illustrates an alternative variation where a single needle may be utilized with one or two ultrasound transducers.

FIG. 5 illustrates yet another alternative variation where three or more needles may be utilized with at least two ultrasound transducers in an alternating manner.

FIGS. 6A and 6B show variations for positioning of the needles and transducers relative to one another.

FIGS. 7A to 7C show side and end views, respectively, of one variation of a distal end portion which may be configured to include an expandable balloon.

FIGS. 8A and 8B show side and end views, respectively, of another variation of a distal end portion which may be configured to include a reconfigurable wire or ribbon member.

FIG. 9 shows an alternative configuration of the ultrasound and infusion assembly which may optionally utilize an RF generator and/or an optional cooling fluid reservoir assembly.

FIG. 10 shows another variation used to deliver laser energy as well as for providing an infusion or injection of a fluid directly into the tissue being treated.

FIGS. 11A to 11C show yet another variation where optical fibers may be placed through the elongate shaft and optionally advanced through the one or more needles protruding from a side surface or from a distal tip of the elongate shaft.

FIG. 12A shows a detail cross-sectional view of an advanceable needle defining a central lumen through which an optical fiber may be positioned.

FIG. 12B shows a detail cross-sectional view of another variation where a lumen opening may terminate along a surface of the needle proximal to a piercing tip.

FIG. 12C shows yet another variation where the optical fiber may be positioned along an outer surface of the piercing needle.

FIGS. 13A to 13C illustrate one method in which an infusion needle may be advanced into the tissue to be treated to infuse a fluid and then an optical fiber may be advanced through the needle to treat the region of tissue via laser energy.

FIG. 14 shows one example for accessing the Eustachian tube via the car canal.

FIG. 15 shows another example for accessing the Eustachian tube via the nasopharyngeal opening.

FIG. 16 shows an example for approaching the nasopharyngeal opening via advancement through the nasal cavity.

FIG. 17 shows another example for approaching the nasopharyngeal opening via advancement through the mouth.

FIGS. 18A to 18C illustrate one variation of a stent deployment assembly which may be used to treat the tissues in and/or around the Eustachian tube and to also deploy a stent therein.

FIGS. 19A and 19B illustrate the assembly of FIGS. 18A to 18C used to deploy the stent within the Eustachian tube.

### DETAILED DESCRIPTION OF THE INVENTION

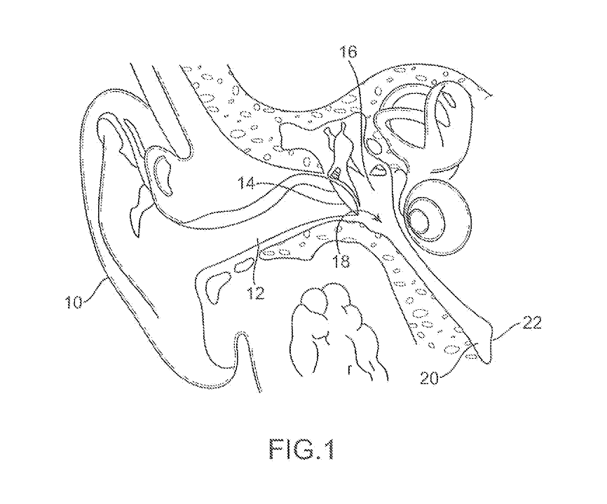

As shown in FIG. 1, the ear canal 12 extends from the external ear or pinna 10 to the tympanic membrane 14. The middle car cavity 16 is located on the other side of the tympanic membrane 14 and opens via Eustachian tube opening 18 to the Eustachian tube 20, which extends to the nasopharyngeal opening 22 located in the pharynx posterior to the mouth and nasal cavity. By reducing the size of tissue, particularly sub-mucosal tissue, in and/or around the Eustachian tube 20, obstruction of the Eustachian tube lumen can be reduced to improve the passage of air and fluid therethrough. One method for reducing the tissue involves the application of energy to the tissue regions beneath the surface of the tissue, e.g., ultrasound, laser energy, etc.

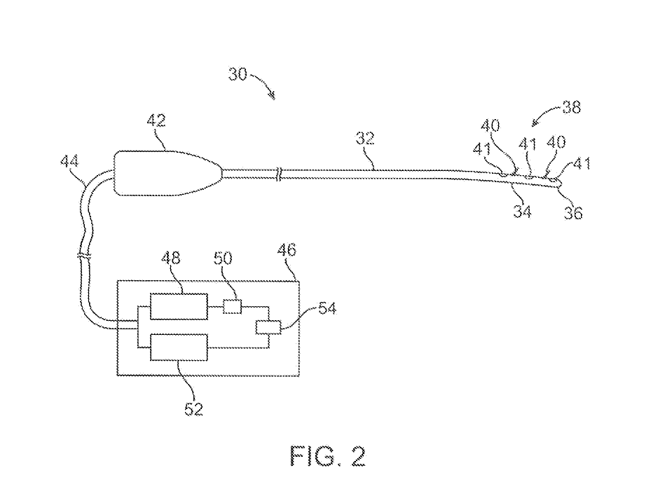

FIG. 2 illustrates a variation of a treatment instrument which may be used to deliver ultrasound energy for treating the tissues underlying the tissue surface as well as for providing an infusion or injection of a fluid directly into the tissue being treated by ultrasound energy. The fluid injected into the tissue may serve a number of different purposes. One purpose is to bulk up the physical size of the tissue by injecting the fluid to present a larger surface area to the ultrasound transducers positioned along the instrument. The enlarged surface area may help to ensure that the ultrasound energy is properly delivered directly into the intended tissue rather than surrounding tissues. Examples of fluids which may be used for bulking the tissue may include any number of suitable fluids, e.g., saline, water, etc.

Another purpose is for drug delivery directly into the treated tissue. For instance, anesthetic fluids or other fluids infused with analgesics (e.g., lidocaine with or without epinephrine, marcaine with or without epinephrine, etc.) may be injected into the tissue to provide for pain management during and after the application of the ultrasound energy. Additionally, other drugs for injection may include any number of medications, such as steroidal drugs (e.g., corticosteroids, dexamethasone, beclomethasone, etc.), non-steroidal drugs (e.g., non-steroidal anti-inflammatory drugs, etc.), anti-inflammatory drugs, anti-histamines (e.g., diphenhydramine, etc.), anti-bacterial drugs, etc. which may be injected to control excessive postoperative swelling as well as infection.

Yet another purpose may be to utilize the one or more injection needles as a positioning tool for ensuring that the ultrasound energy, which is directional, is delivered into the intended tissue. For example, the injection needle(s) may be initially positioned directly within the tissue prior to application of the ultrasound energy since the ultrasound transducer(s) along the probe may be aligned with the injection needle(s). Accordingly, if the needle(s) is positioned directly within the tissue to be treated, the operator may be assured that the ultrasound energy will be directionally aligned with the appropriate tissue region.

Returning now to FIG. 2, ultrasound and infusion probe 30 is illustrated as having an elongate shaft 32 with a distal end portion 34 having a rounded or blunted atraumatic, tip 36 to prevent trauma to contacted tissue. Elongate shaft 32 may have a length which is sufficient to enable the insertion of distal end portion 34 into the Eustachian tube 20 of a patient. Accordingly, the length of shaft 32 may range any where from several centimeters to 25 cm or longer while the distal end portion may range anywhere, e.g. from 10 to 30 mm in length or longer if so desired. The elongate shaft 32 itself may be conformed to any cross-sectional area so long as the overall size is sufficient to allow for insertion and advancement into the Eustachian tube 20 and against the appropriate tissue surface. Moreover, elongate shaft 32 may optionally define one or more visual markings or indicators along its length to indicate a depth of the shaft 32 into the Eustachian tube 20 by comparison against an anatomical landmark.

The distal end portion 34 may be angled relative to the elongate shaft 32 or it may be straight depending upon the desired configuration. The distal end portion 34 may have an end effector assembly 38 which has one or more hollow infusion/injection needles 40 which are retractably disposed within the distal end portion 34. During advancement into the lumen and positioning against the tissue, the infusion/injection needles 40 may be positioned within the distal end portion 34 so as to present a smooth atraumatic surface to the tissue. When a fluid is to be injected into the tissue after the probe 30 has been desirably positioned against the tissue surface, a control or advancement mechanism on handle 42, which is connected to a proximal end of shaft 32, may be actuated to advance needles 40 at least partially out of distal end portion 34.

The illustration of FIG. 2 shows two retractable infusion/injection needles 40; however, fewer or additional needles 40 may be utilized depending upon the desired results and procedure to be undertaken. Between or adjacent to needles 40 are positioned, one or more ultrasound transducers 41 along the body of distal end portion 34. The illustration shows three ultrasound transducers for delivering the ultrasound energy, but fewer or additional transducers 41 may be utilized or positioned along the distal end portion 34.

An electronic/fluid cable 44 is electrically and fluidly connected to handle 42 and is further connected to a power/infusion assembly 46. Within assembly 46 is a fluid reservoir 48 and a pump 50 electrically coupled to controller or central processor 54. Any of the above-mentioned fluids, e.g., analgesics, anesthetics, anti-inflammatory drugs, water, saline, etc., may be filled within reservoir 48 for delivery through cable 44, elongate shaft 32 and through the one or more infusion/injection needles 40 for delivery into the tissue. The infusion rate of the fluid and control of the pump 50 may be determined by the controller 54. An example of a pump which is pre-programmed to inject a fluid in a controlled injection rate and which may be utilized with the pump 50 is commercially available as the CompuDent® delivery system and Wand® handpiece (Milestone Scientific, Inc., South Orange Livingston, N.J.). Power supply 52 may also be provided within assembly 46 and may be controlled by controller 54 to control the amount of energy provided by the ultrasound transducers 41 located in distal end portion 34.

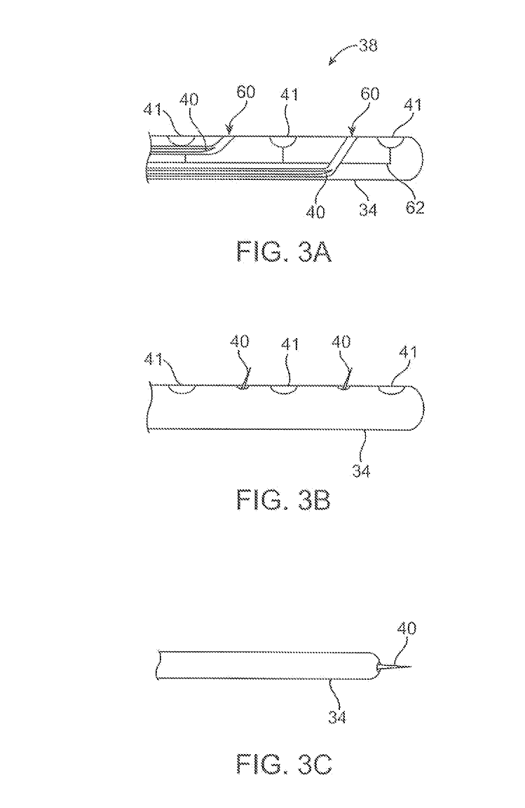

As mentioned above, during delivery and positioning of elongate shaft 32 against the tissue, the one or more needles 40 may be retracted within distal end portion 34, as shown in the partial cross-sectional detail view of FIG. 3A. As illustrated, infusion/injection needles 40 may be positioned within their respective needle lumens 60 positioned between the ultrasound transducers 41. The piezoelectric transducers of each of the ultrasound transducers 41 may be electrically coupled via wires 62 routed through elongate shaft 32 to the power supply 52 located within assembly 46. The piezoelectric transducer may be vibrated over a range of frequencies, e.g., anywhere from 0.5 to 12 MHz, or more typically between 5 to 12 MHz, to generate the ultrasound energy to treat the tissue.

When the infusion/injection needles 40 are to be deployed into or against the tissue, they may be advanced distally through needle lumens 60 until they project from a surface of the elongate shaft 32, as shown in FIG. 3B. Needles 40 may be configured to project from the shaft from less than 1 mm to more than 2 mm or anywhere therebetween provided that needles 40 are able to sufficiently contact against and/or into the tissue surface to inject the fluid. Another variation is shown in the side view of FIG. 3C, which illustrates a configuration where needle 40 may be retractably projected from the distal end of shaft distal portion 34.

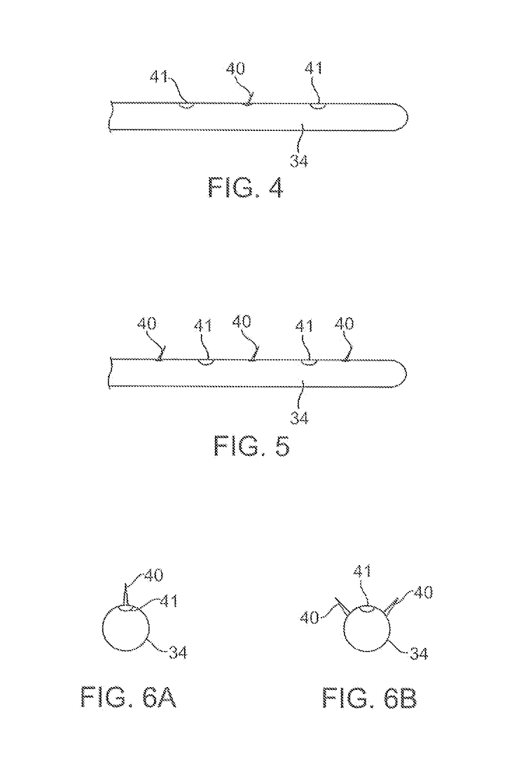

The configuration and number of infusion/injection needles 40 and ultrasound transducers 41 may be further varied depending upon the desired effect. FIG. 4 illustrates an alternative variation where a single needle 40 may be utilized with one or two ultrasound transducers 41. Alternatively, FIG. 5 shows a variation where three or more needles 40 may be utilized with at least two ultrasound transducers 41 in an alternating manner. Moreover, the circumferential positioning of the needles 40 relative to the transducers 41 may also be varied. FIG. 6A shows one variation where each of the needles 40 and transducers 41 may be aligned linearly while FIG. 6B shows another variation where two or more needles 40 may be off-set to project at an angle relative to one another with the ultrasound transducer 41 positioned therebetween.

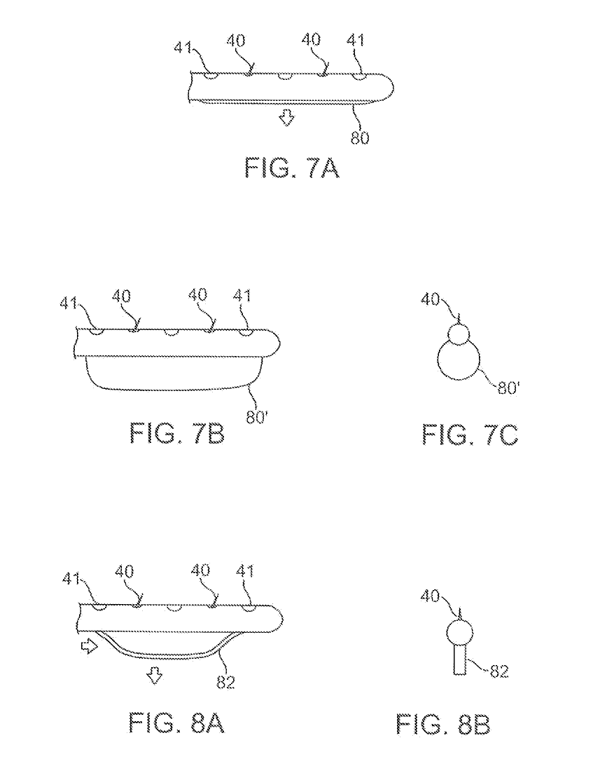

In alternative configurations, the distal end effectors may include a mechanism for securely pressing the surface of the elongate shaft against the tissue surface to be treated to ensure piercing of the needles into the tissue as well as sufficient contact for the ultrasound transmission. For instance, FIG. 7A illustrates one variation of a distal end portion which may be configured to include an expandable balloon 80. Once the shaft has been desirably positioned against the tissue surface, balloon 80′ may be expanded via a fluid such as water or saline or a gas such as air delivered through an inflation lumen defined through shaft 32, as shown in FIGS. 7B and the end view in FIG. 7C. Once the desired treatment has been completed, balloon 80′ may be deflated and the elongate shaft 32 may be moved to another region of the tissue or removed entirely.

Another variation of a mechanism is shown in the side and end views of FIGS. 8A and 8B, which illustrate a wire or ribbon member 82 which may be reconfigured from a low-profile configuration against the elongate shaft 32 to an expanded shape, as shown. When the elongate shaft 32 is to be securely presented against the tissue surface, wire or ribbon member 82 may be advanced or actuated from handle 42 to urge the member 82 into a reconfigured and expanded shape to push against the tissue.

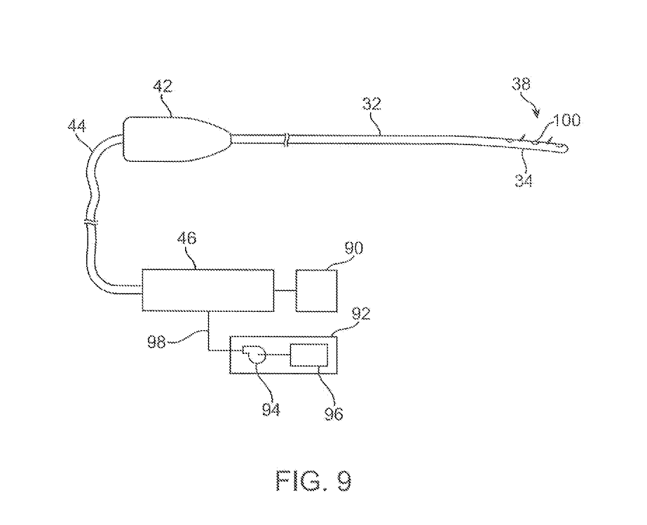

In yet another configuration shown in FIG. 9, the ultrasound and infusion probe 30 may optionally include an additional radio-frequency energy generator 90, which may be configured to deliver RF energy to one or more needles to ablate the pierced tissue. Ablation of the pierced regions of tissue may help to coagulate the pierced tissue. Moreover, the ultrasound and infusion probe 30 may also optionally include a cooling unit 92 fluidly connected via fluid line 98 to power/infusion assembly 46. Cooling unit 92 may comprise a pump 94 fluidly coupled to a reservoir 96 containing cooled or chilled fluid 96, e.g., saline, water, etc. The cooled fluid 96 may be fluidly connected through elongate shaft 32 to a cooling fluid port 100 positioned along distal end portion 100. Before, during, or after ultrasound energy transmission into the tissue, the cooled fluid may be pumped from reservoir 96 through cooling fluid port 100 to cool the surface of the tissue to ensure that the tissue surface is unperturbed by the energy applied beneath its surface.

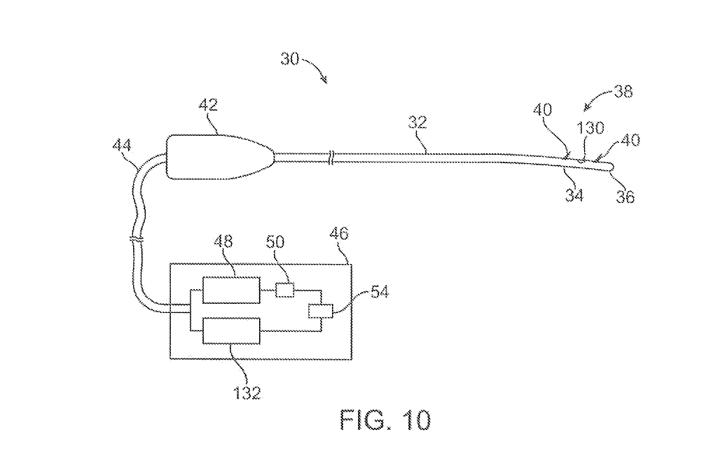

FIG. 10 shows yet another variation used to deliver laser energy as well as for providing an infusion or injection of a fluid directly into the tissue being treated. The probe assembly may include a laser generator 132 for delivering laser energy through the probe shaft 32 to the distal end portion 34, e.g., via optical fibers positioned through the instrument with the terminal end of the optical fiber placed between or adjacent to the needles 40.

In use, the instrument may be delivered and positioned adjacent to the tissue to be treated. During or after the injection of the needles 40 and delivery of fluids in the tissue, the laser generator 132 may be actuated to deliver laser energy through the terminal end of the optical fiber 130. The laser may be configured as any number of laser instruments. For instance laser generator 132 may be an Argon laser or CO₂ laser capable of generating laser temperatures, e.g., of 750° to 900° C., to vaporize the underlying tissue.

Moreover, controller 54 may be configured to control laser generator 132 to deliver pulsed laser energy through fiber terminal end 130 for a controlled period of time and frequency.

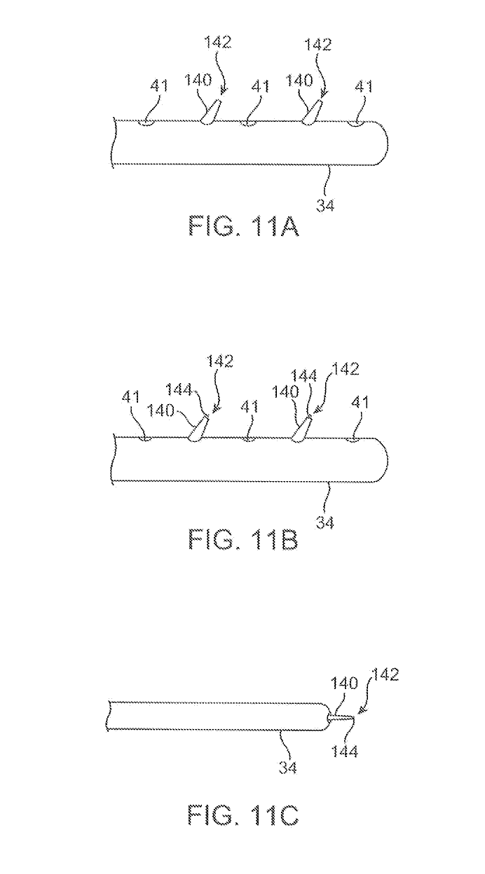

Another variation for delivering laser energy for tissue treatment is illustrated in the detail side views of FIGS. 11A and 11B. As shown, needles 140 may each define a lumen 142 therethrough, e.g., for infusion of fluids as described above, and/or advancing one or more corresponding optical fibers 144 directly into and through the needles 140, as shown in FIG. 11B. The optical fiber 144 may be advanced through the lumen 142 of the needle 140 after or during infusion of the fluid into the tissue. Alternatively, the infusion of fluids may be omitted entirely and the optical fiber 144 may be advanced through the needle 140 after or simultaneously with the needle 140 when projected from the shaft 34 into the tissue to be treated. In the variation shown in FIG. 11C, with needle 140 retractably projecting from the distal end of the shaft, optical fiber 144 may be advanced through lumen 142, as above.

Moreover, the laser energy passed through the optical fibers 142 may be utilized in conjunction with the ultrasound energy delivered via the one or more ultrasound transducers 41, as above, or alone.

In passing the optical fiber 144 through the needle body, the fiber 144 may be independently translatable within the needle lumen 142. In this variation, the fiber 144 may be passed through the same lumen utilized for fluid infusion through the needle, if fluid infusion is utilized. Alternatively, the optical fiber 144 may be affixed within the lumen 142 of the needle such that advancement or retraction of the needle also likewise advances or retracts the optical fiber 144 relative to the elongate shaft 34. Moreover, the optical fiber 144 in either case may be configured (if affixed) or otherwise urged (if translatable) to extend just proximal to, adjacent with, or distally beyond the lumen opening or needle tip and into the tissue during treatment.

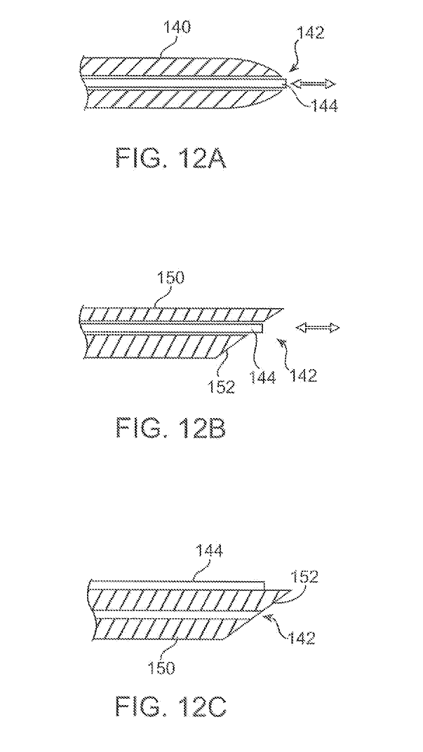

FIGS. 12A illustrates one variation of optical fiber 144 positioned within lumen 142 of needle 140 in the partial cross-sectional detail view. Optical fiber 144 may be optionally translatable relative to needle 140, as indicated by the arrow, and positioned centrally through needle 140 such that the distal tip of optical fiber 144 is extendable through the distal tip of needle 140.

FIG. 12B illustrates another variation where optical fiber 144 may be optionally translatable, as indicated by the arrow, relative to needle 150 which defines an angled piercing surface 152. Optical fiber 144 may exit the lumen opening at a location along a side surface of the needle 50 proximal to the piercing tip so as not to inhibit entry of the needle 150 into the tissue to be treated.

FIG. 12C illustrates yet another variation where optical fiber 144 may be optionally placed or integrated along an outer surface of the needle 150 leaving lumen 142 open for fluid infusion. Optical fiber 144 it may be adhered or affixed, to needle 150 via any number of mechanisms, e.g., adhesives, outer sheath, etc., or otherwise integrated with the body of needle 150.

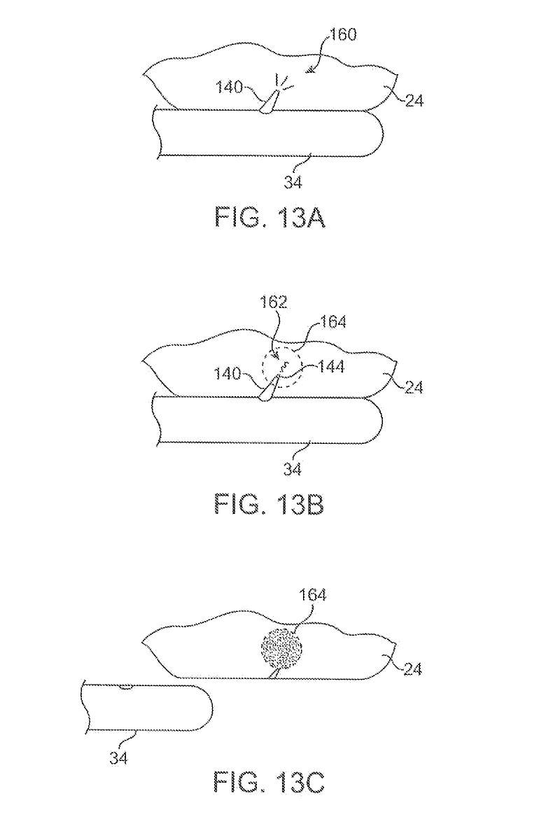

In an exemplary method of use, the elongate shaft 34 may be advanced with the needles in their retracted position within shaft 34 and placed against the region of tissue to be treated, as shown in FIG. 13A. One or more needles 140 (a single needle is shown for illustrative purposes only and is not intended to be limiting) may be advanced from shaft 34 and into the tissue. Once desirably positioned, fluid 160 may be optionally infused into the tissue, as described above, and optical fiber 144 may be advanced through needle 140. Alternatively, optical fiber 144 may be advanced into the tissue 24 simultaneously with needle 140 and infusion of the tissue may be omitted.

With optical fiber 144 positioned proximate to or within the tissue 24, laser energy 162 may be passed through optical fiber 144, as described above, to ablate the tissue region 164 around the needle 140, as shown in FIG. 13B. Optionally, ultrasound energy may be applied prior to, during, or after application of the laser energy depending upon the desired results. Once the treatment has been completed, needle 140 and optical fiber 144 may be retracted or withdrawn and elongate shaft 34 may be repositioned to another tissue region or withdrawn entirely from the patient body, as shown in FIG. 13C.

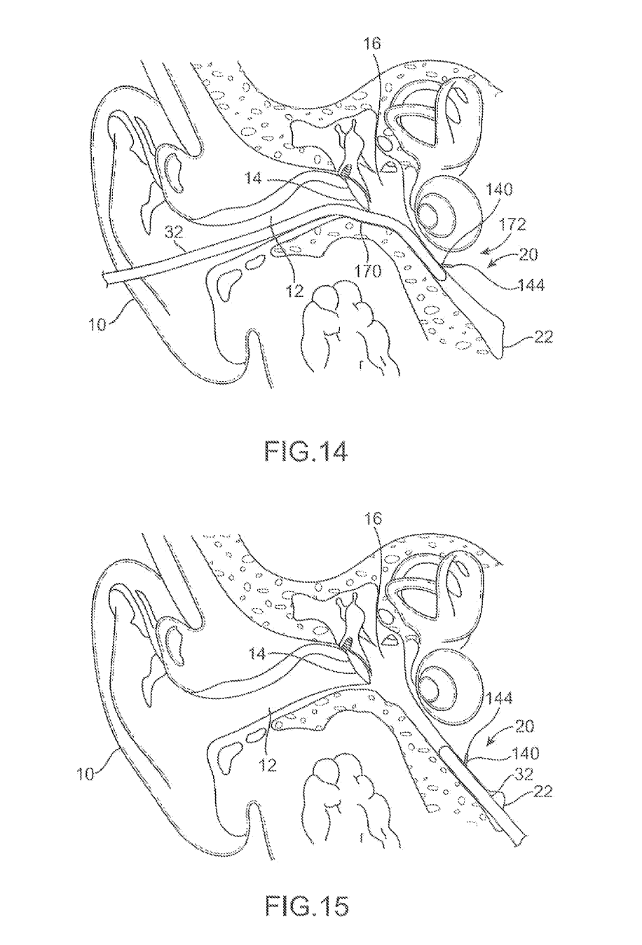

One particular example for accessing regions within the Eustachian tube 20 to treat the tissue defining tile lumen is shown in the illustration of FIG. 14. Shaft 32, sized appropriately for entry into the Eustachian tube 20, may be advanced through the ear canal 12 and through an opening drilled through the bone 170 and/or fibrous annulus surrounding the tympanic membrane 14. Such an opening may be drilled with a surgical drill. Once the distal end effector of shaft 32 is desirably advanced through the middle ear cavity 16 and appropriately positioned within the Eustachian tube 20, one or more needles 140 may be projected from the shaft 32 and inserted into the surrounding tissue such that the one or more optical fibers 144 may be advanced through the lumen of the needle 140 and into the sub-mucosal tissue 172. With the optical fiber 144 placed within or proximate to the tissue and structurally supported via the needle 140, laser energy may be applied to the tissue, as described above. Optionally, ultrasound energy may also be applied additionally or in place of the laser energy with or without infusing any fluids or therapeutic drugs, as desired. Once an initial area has been treated, the needle 140 and optical fiber 144 may be retracted back into the shaft 32 and the distal end effector may be moved to another area of tissue within or around the Eustachian tube 20 for treatment or the shift 32 may be withdrawn entirely from the patient.

Aside from insertion through the ear canal 12, the shaft 32 and distal end effector may also be advanced through the mouth and/or nose of the patient and inserted through the nasopharyngeal opening 22 of the Eustachian tube 20. As illustrated in FIG. 15, once the distal end effector of the shaft 32 has been appropriately situated within or around the Eustachian tube 20, the one or more needles 140 may be projected and inserted into the surrounding tissue and laser treatment via optical fiber 144, in combination with or in place of any of the alternative energy and infusion delivery modalities described above, may be accomplished.

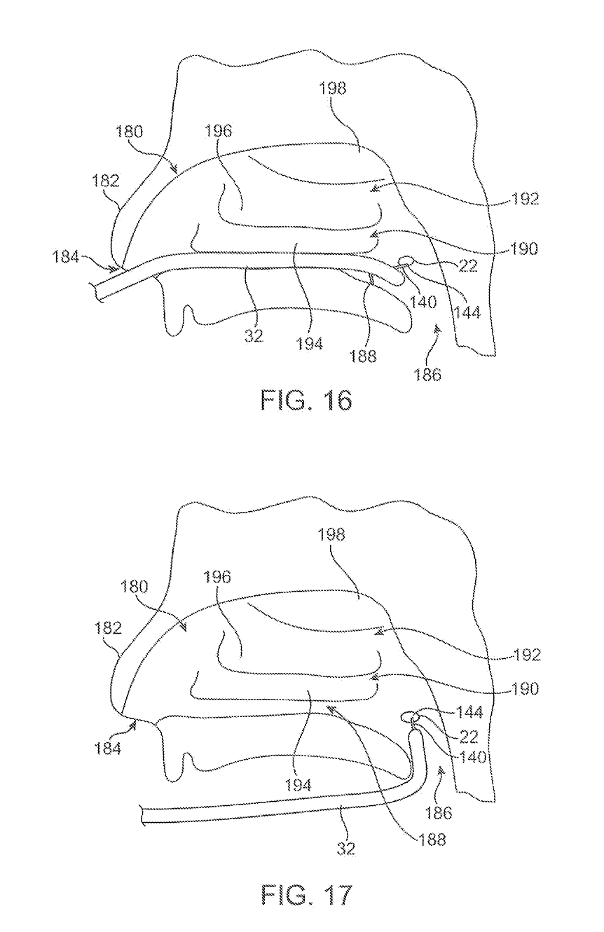

As shown in FIG. 16, the sinus cavity 180 which can become obstructed include the nasal passageways leading from the nose 182 to the pharynx 186. Tile nasal airway has several compartments, namely the inferior 188, middle 190, and superior 192 nasal meatus. The turbinates, also referred to as nasal concha, are a series of tissues which form at least a portion of these nasal compartments 188, 190, 192. Forming a portion of the inferior nasal meatus 188 is the inferior nasal turbinate 194. The inferior 194 and middle nasal turbinate 196 each from a portion of the middle nasal meatus 190. The middle 196 and superior nasal turbinate 198 each form a portion of the superior nasal meatus 192. When the inferior 194, middle 196 and/or superior nasal turbinate 198 become enlarged, the various nasal meatus which allow air to pass through the nostril 184 into the pharynx 186 can become obstructed.

FIG. 16 illustrates an example of advancement of shaft 32 through the nasal cavity 180 by inserting the shaft 32 into the nose 182 through a nostril 184 and advancing the shaft 32 towards the pharynx 186. The shaft 32 may be advanced through one of the passageways, e.g., inferior 188, middle 190, or superior 192 nasal meatus, defined respectively by the inferior 194, middle 196, or superior 198 nasal turbinates. Once the distal portion of shaft 32 is in proximity to the nasopharyngeal opening 22 of the Eustachian tube 20, the shaft 32 may be inserted, as above or needle 140 may be projected and optical fiber 144 may be advanced into Eustachian tube 20 to effect treatment or positioned proximate to or against the surrounding tissue for treatment.

FIG. 17 illustrates another example in which shaft 32 may be advanced through the mouth of the patient and towards the nasopharyngeal opening 22, as illustrated. Once the distal end portion of shaft 32 is proximate to the opening 22, shaft 32 may be advanced directly into the Eustachian tube 20, as shown above, or the one or more needles 140 and optical fibers 144 may be projected directly into Eustachian tube 20 or used to treat the tissues surrounding the opening 22. Any of the variations of shaft 32 may be utilized, although a distally projecting needle 140 is illustrated in this example.

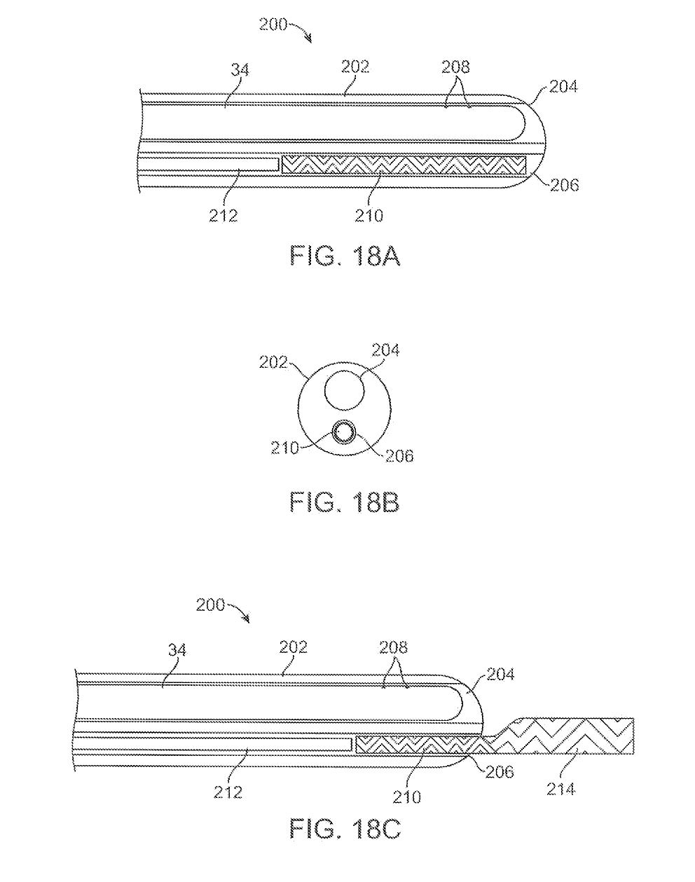

Additionally, although the treatment instrument may be utilized alone in treating the tissues in or around the Eustachian tube 20, it may also be utilized in combination with a stent deployment assembly, as illustrated in the partial cross-sectional and end views of FIGS. 18A and 18B, respectively. In certain patients, it may be desirable to not only treat the tissues within or around the Eustachian tube 20, but it also may be desirable to implant a stent within the Eustachian tube 20 to maintain patency of the lumen. As shown, one example of a stent delivery assembly 200 is illustrated having an outer sheath 202 which defines an instrument lumen 204 and a stent lumen 206. Although the shaft portion 34 with its one or more needles retracted within needle openings 208 is illustrated as positioned within lumen 204, other instruments (e.g., energizable probes, visualization instruments such as endoscopes, etc.) may be positioned within the lumen 204 depending upon the desired treatment.

An expandable stent 210 appropriately sized for placement within the Eustachian tube 20 may be disposed in a low-profile configuration within lumen 206, distal to a pusher mechanism 212, which may be actuated via the user from a proximal end of the assembly to urge the stent 210 distally out from lumen 206. Stent 210 may be fabricated and configured utilizing any number of stents known to one of ordinary skill and configured from a shape memory alloy, such as a nickel-titanium alloy. It may comprise a bare scaffold or it may alternatively comprise a covering. In either case, stent 210 may also comprise a valve within such that air or fluids may be passed through when desired but remains closed otherwise. Some examples of stents which may also be utilized within sheath 202 are further shown and described in, e.g., U.S. Pat. No. 4,015,607; U.S. Pat. No. 6,589,286; and US 2005/0240147 A1, each of which is incorporated herein by reference in its entirety.

When assembly 200 is advanced into the Eustachian tube 20 of a patient, ablation treatment may be performed upon the tissue using the treatment instrument. During or after the treatment, stent 210 may be deployed from lumen 206 by advancing pusher 212 distally to urge the stent 210 out from lumen 206 and into Eustachian tube 20. When free from the constraints of lumen 206, stent 210 may begin to self-expand (or it may be expanded via an expanding mechanism such as an inflatable balloon) into an expanded stent 214, as shown in FIG. 18C. Moreover, during stent deployment, shaft 34 may be withdrawn from lumen 204 and a visualization instrument may be disposed within the lumen 204 to visualize the deployment of the stent.

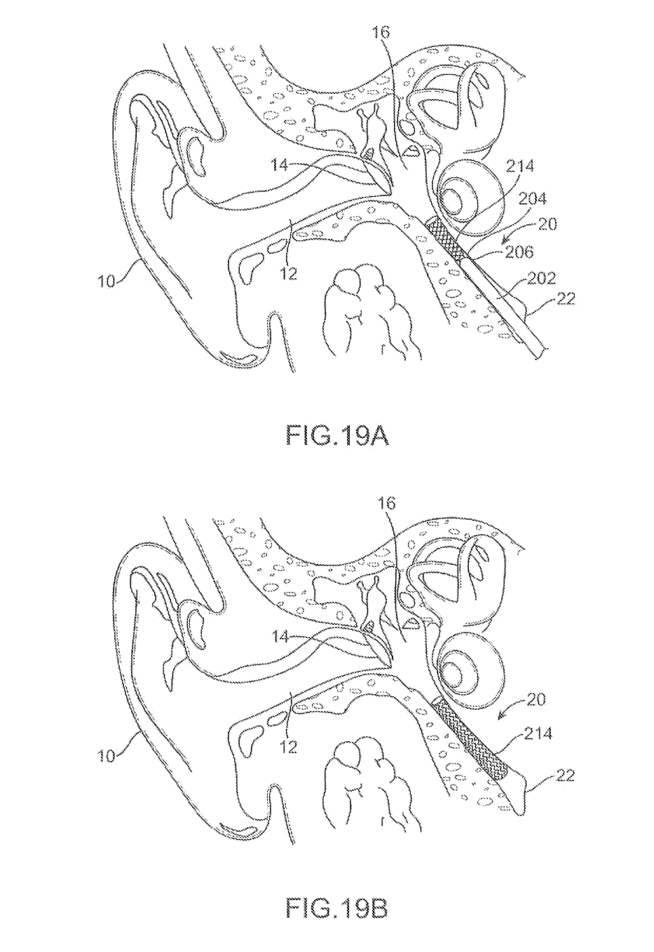

FIG. 19A illustrates one example for deploying a stent within the Eustachian tube 20 of a patient via sheath 202, which may be advanced either through the ear canal 12 or via the nasopharyngeal opening 22, as above. In either case, once the sheath 202 has been desirably positioned, the tissue in and/or around Eustachian tube 20 may be treated via the treatment shaft 32, as described above. If desired or necessary, the stent may then be urged, via pusher 212 to begin the deployment of stent 214 into its expanded profile as shown. Outer sheath 202 may be withdrawn slowly while stent 214 is deployed. Once fully deployed, stent 214 may be tell within Eustachian tube 20 to maintain its patency and sheath 202 may be withdrawn from the patient, as shown in FIG. 19B.

The applications of the devices and methods discussed above are not limited to the treatment of the tissue regions in or around the Eustachian tube but may include any number of further treatment applications. Modification and combinations of the above-described assemblies and methods for carrying out the invention, and variations of aspects of the invention that are obvious to those of skill in the art arc intended to be within the scope of the claims.

What is claimed is:

1. A method of treating tissue in or around a Eustachian tube of a patient body via laser energy, comprising:
positioning an elongate shaft having a distal end against a tissue region of interest in or around the Eustachian tube;
piercing the tissue region via at least one needle retractably disposed near or at the distal end;
advancing at least one optical fiber terminal end into the tissue region within or along the at least one needle; and
applying laser energy via the at least one optical fiber to the tissue region.

2. The method of claim r wherein positioning comprises advancing the elongate shaft through a nostril of a patient and into or against a nasopharyngeal opening.

3. The method of claim 1 wherein positioning comprises advancing the elongate shaft through a mouth of a patient and into or against a nasopharyngeal opening.

4. The method of claim 1 wherein positioning comprises advancing the elongate shaft through an ear canal of a patient and into the Eustachian tube.

5. The method of claim 1 wherein piercing comprises piercing the tissue region via a plurality of needles.

6. The method of claim 1 wherein piercing further comprises advancing the at least one needle from within the elongate shaft to project externally of a surface of the elongate shaft.

7. The method of claim 1 further comprising infusing or injecting a fluid through the at least one needle into the tissue region prior to applying laser energy.

8. The method of claim 7 wherein infusing or injecting comprises infusing or injecting a fluid selected from the group consisting of anesthetics, analgesics, anti-inflammatory drugs, anti-histamines, non-steroidial drugs, steroidal drugs, anti-bacterial drugs, water, and saline.

9. The method of claim 1 wherein applying comprises transmitting laser energy via a plurality of optical fiber terminal ends positioned near or at the distal end.

10. The method of claim 1 further comprising applying a cooling fluid onto the surface of the tissue region.

11. The method of claim 1 further comprising applying ultrasound energy to the tissue region via one or more ultrasound transducers positioned near or at the elongate shaft distal end.

12. The method of claim 1 further comprising deploying an expandable stent into or against the tissue region within the Eustachian tube.

13. The method of claim 1 further comprising applying RF energy to the tissue region.

14. A Eustachian tube treatment system, comprising:
an outer sheath sized for advancement within the Eustachian tube and defining at least an instrument lumen and a stent lumen;
an elongate shaft having a distal end, a proximal end, and a length therebetween positionable within the instrument lumen;
at least one needle retractably positioned to extend from a surface of the shaft near or at the distal end;
at least one optical fiber positioned within or along the at least one needle such that a distal end of the optical fiber is movable with respect to the shaft; and
an expandable stent removably positioned within the stent lumen.

15. The system of claim 14 wherein the outer sheath is sized for advancement within the Eustachian tube.

16. The system of claim 14 further comprising a laser generator in communication with the at least one optical fiber.

17. The system of claim 14 further comprising at least one ultrasound transducer positioned along the shaft.

18. The system of claim 14 further comprising a fluid reservoir in communication with the at least one needle.

19. The system of claim 14 further comprising an RF energy generator in communication with the at least one needle.
